(19) United States
(12) Patent Application Publication
(10) Pub. No.: US 2023/0034893 A1
(43) Pub. Date: Feb. 2, 2023

(54) PROCESS FOR PRESERVING THE HUSK AND USE OF RAMBUTAN (NEPHELIUM LAPPACEUM) IN FOOD PRODUCTS

(71) Applicant: Rambuhealth Corp., New York, NY (US)

(72) Inventor: Hider Rojas, New York, NY (US)

(73) Assignee: Rambuhealth Corp., New York, NY (US)

(21) Appl. No.: 17/966,924

(22) Filed: Oct. 17, 2022

Related U.S. Application Data
(62) Division of application No. 17/364,996, filed on Jul. 1, 2021.
(60) Provisional application No. 63/047,113, filed on Jul. 1, 2020.

(51) Int. Cl. A61K 36/77; A01N 3/00; A23L 33/105; A23L 2/52

(52) U.S. Cl. CPC A61K 36/77; A01N 3/00; A23L 33/105; A23L 2/52; A61K 2236/15; A61K 2236/51; A61K 2236/331; A61K 2236/55

(57) ABSTRACT

A formulation for delivering one or more health benefits to a human post-consumption and a method to create the formulation are described. The formulation comprises a shell extract of Nephelium lappaceum in a form of a powder or a liquid and at least one material. The at least one material comprises a polysaccharide, a protein, a lipid, and/or a food grade compound. The one or more health benefits to the human include support or regulation of blood sugar, blood pressure, body weight, performance/recovery from sports, immune health, anti-aging, pain management, and/or joint health.

### CROSS-REFERENCE TO RELATED APPLICATIONS SECTION

This Application is a U.S. Non-Provisional Patent Application and divisional of U.S. Non-Provisional patent application Ser. No. 17/364,996 filed on Jul. 1, 2021 that claims priority to U.S. Provisional Patent Application Ser. No. 63/047,113 filed on Jul. 1, 2020, the entire contents of which are hereby incorporated by reference in their entirety.

### FIELD OF THE EMBODIMENTS

The field of the invention and its embodiments relate to formulations for delivering one or more health benefits to a human post-consumption. More specifically, the field of the invention and its embodiments relate to formulations for delivering the one or more health benefits to a human that comprises a shell extract of Nephelium lappaceum (or rambutan) in a form of a powder or a liquid and at least one material (e.g., encapsulating material). The at least one material comprises a polysaccharide, a protein, a lipid, and/or a food grade compound. The one or more health benefits to the human include support or regulation of blood sugar, blood pressure, body weight, performance/recovery from sports, immune health, gut health, neuroprotectice effect, anti-aging, pain management, and/or joint health.

### BACKGROUND OF THE EMBODIMENTS

Antioxidants, compounds that inhibit oxidation, such as vitamin C, vitamin E, carotene, lycopene, lutein, and many other substances, play a role in helping to prevent diseases, such as cancer, cardiovascular disease, Alzheimer's disease, and macular degeneration. Antioxidants are thought to prove helpful, as they can neutralize free radicals, which are toxic byproducts of natural cell metabolism. The human body naturally produces antioxidants, but the process isn't completely effective and that effectiveness declines with age. Research is increasingly showing that those who eat antioxidant-rich foods, such as a berries, beans, fruits, vegetables, etc., reap health benefits. However, there is a need for a new composition to provide these antioxidant benefits to humans.

### Review of Related Technology

CN 109232119 A describes a green organic fertilizer containing bunge pricklyash leaf The green organic fertilizer includes, among other components, a water-retaining agent. The water-retaining agent is mainly prepared from rambutan husk, cicada pupa powder, chamomile, lemon pericarp, banana pericarp, fly maggot powder, common waterhyacinth herb, and pomegranate pericarp.

CN 109265277 A describes a green organic fertilizer to prevent and treat corn root rot. The green organic fertilizer comprises, by weight, 5-6 parts of fermented fruit peel, among other components. The fermented fruit peel is mainly made with apple peel, pawpaw peel, watermelon peel, peel of citrus reticulata ‘Shiranui’, lemon peel, rambutan peel, and fig peel.

CN 109265278 A describes a green organic fertilizer for preventing corn stalk soft rot. The green organic fertilizer comprises, by weight, 5-6 parts of a fruit peel fermented material, among other components. The fruit peel fermented material is mainly prepared from apple peel, pawpaw peel, watermelon peel, tangerine peel, lemon peel, rambutan peel, and fig peel.

KR 1020200034954 A, KR 1020200035040 A, CN 110691630 A, and CN 110944720 A describe cosmetic use of a pericarp extract of the plant Nephelium lappaceum in order to maintain and/or increase the hydration and/or the barrier effect of the skin and/or of the mucous membranes.

KR 1020200002922 A describes uses of various extracts of the Nephelium lappaceum plant for increasing the firmness and/or elasticity of the skin and/or mucous membranes, by increasing type I and/or type V collagen and/or LOX-L, fibulin-5, emiline-1 and/or fibrillin-1 gene and/or protein expression, and/or by decreasing CYR61 expression, in the skin and mucous membranes.

CN 110037949 A describes use of an extract of the plant Nephelium lappaceum to strengthen the hair follicle, by maintaining and/or increasing the expression of type V collagen and/or fibrillin-1 and/or by increasing proliferation of hair follicle fibroblasts and/or cell viability and/or ATP synthesis and/or mitochondrial activity and/or by decreasing cell damage and/or cell senescence. This reference also relates to the use of an extract of Nephelium lappaceum in a cosmetic composition comprising at least one cosmetically acceptable excipient.

KR 102086166 B 1and KR 1020190066478 A describe an antimicrobial composition including the peel extract of rambutan (Nephelium lappaceum) fruits and, more specifically, to an antimicrobial composition with excellent antimicrobial activity by including the hexane fraction or ethyl acetate fraction of rambutan peel extract. The rambutan peel extract of the present invention shows excellent antimicrobial activity and accordingly, can be used in an antimicrobial pharmaceutical composition, health functional food, a cosmetic composition, an animal feed composition, food, additives for cosmetics or animal feed, various cleaning agents, and various detergents. Moreover, the rambutan peel extract of the present invention can be used beneficially in a pharmaceutical industry, a food industry, a livestock industry, a health industry, and a cosmetic industry by being easily processed into various forms such as liquid, cream, powder, ring, and pill since heat stability is excellent and the loss of activity does not appear in plasma and under an acid condition of pH 2.

KR 1020190066460 A describes an anti-diabetes composition that includes the seed extract of rambutan fruit (Nephelium lappaceum) and, more specifically, to an anti-diabetic composition with excellent inhibitory activity of starch decomposition enzymes by including the ethanol extract of rambutan seeds.

WO 2018/150395 A2, WO 2018/150395 A3, and WO 2018/150395 A4 describe a medical or cosmetic formulation (such as pastes, gels, lotions, serums, creams, peels, and/or sprays) comprising 4-n-butylresorcinol in a concentration greater than substantially 18%, and preferably between substantially 20% and substantially 60%, and diluted with a hydrophilic agent. The formulation may be used for treating various skin conditions, e.g. complexion, hyperpigmentation, melasma, pore size, skin density, hydration, superficial scars, post-operative scars, hypertrophic and atrophic scars, post-inflammatory hyperpigmentation, solar lentigines, acne vulgaris, and/or may function as an anti-aging treatment. The formula may include peels and skincare using various delivery systems. An example formulation of this reference may include rambutan.

CN 108935705 A describes a milk-tea powder, which includes 2-4 parts of rambutan powder, by mass, among other components.

KR 101870952 B1 describes an anti-thrombotic composition comprising a peel extract of rambutan fruits (Nephelium lappaceum) as an active ingredient, and more specifically, to a pharmaceutical composition for preventing or treating/alleviating thrombosis through inhibition of blood coagulation, which comprises, as an active ingredient, a rambutan peel extract prepared from collected peels of rambutan fruits washed with water.

CN 106387910 A relates to a method to prepare a Nephelium lappaceum shell extract. The method comprises the following steps: (1) crushing Nephelium lappaceum fruit shells, (2) pulping the crushed Nephelium lappaceum fruit shells, (3) adjusting the pH of the homogenized pulp with citric acid, (4) performing microwave enzyme deactivation treatment, (5) performing cooling to room temperature, (6) adding sodium bicarbonate for adjusting the pH to a neutral pH, (7) adding cellulase and hemicellulase, (8) stirring and performing enzymolysis, (9) squeezing to obtain juice, and (10) filtering off impurities to obtain a filtrate. Then, the method may include: (11) drying the squeezed residues and crushing the squeezed residues to obtain powder, (12) mixing the Nephelium lappaceum shell powder with an ethanol aqueous solution, (13) placing the mixture into a pressure kettle, (14) performing boiling-water-bath ultrasonic extraction, (15) after the extraction is completed, performing filtration for removing residues and taking the obtained filtrate, (16) performing a separation working procedure to remove ethanol in the filtrate, and (17) merging the filtrate without the ethanol with the filtrate obtained in step (1) so as to obtain the Nephelium lappaceum shell extract.

CN 105167043 A relates to a healthcare Nephelium lappaceum and guava beverage.

WO 2008/066370 A1 relates to a method of extraction of Nephelium lappaceum. The reference also describes use of the extract alone or in combination with other active principals in the nutraceutical, cosmeceutical, and pharmaceutical industries.

KR 1020060007083 A relates to a skin-whitening cosmetic composition comprising at least Nephelium lappaceum.

Various formulations exist in the art. However, these formulations are substantially different from the present disclosure, as the other inventions fail to solve all the problems taught by the present disclosure.

### SUMMARY OF THE EMBODIMENTS

The present invention and its embodiments relate to formulations for delivering one or more health benefits to a human. More specifically, the present invention and its embodiments relate to formulations for delivering one or more health benefits to a human that comprises a shell extract of Nephelium lappaceum (or rambutan) in a form of a powder or a liquid and at least one material (e.g., encapsulating material). The at least one material comprises a polysaccharide, a protein, a lipid, and/or a food grade compound. The one or more health benefits to the human include support or regulation of blood sugar, blood pressure, body weight, performance/recovery from sports, immune health, gut health, neuroprotectice effect, anti-aging, pain management, and/or joint health.

A first embodiment of the present invention describes a method to maintain a freshness of a shell or a husk of Nephelium lappaceum. Such method preserves the shell or the husk during a non-harvest season. The method comprises washing the shell (or the husk) of the Nephelium lappaceum with a first solution (e.g., a neutral soapy solution), washing the shell of the Nephelium lappaceum with a component (e.g., a 100 parts per million chlorine solution, quaternary ammonium compounds, acetic acid, peracetic acid, or other non-toxic disinfectant), and separating the shell of the Nephelium lappaceum from the Nephelium lappaceum. In some examples, the separation of the shell of the Nephelium lappaceum from the Nephelium lappaceum comprises a single cut so as not to compromise an integrity of the shell.

The method further includes: packaging the shell of the Nephelium lappaceum via vacuum packaging or another similar method and utilizing a means to decrease an enzymatic activity and a microbial activity of the packaged shell of the Nephelium lappaceum, while maintaining a moisture loose and acidity ratio. The means to decrease the enzymatic activity and the microbial activity of the packaged shell of the Nephelium lappaceum may include freeze shocking with air, pressurized carbon dioxide, or liquid nitrogen. Next, the packaged shell of the Nephelium lappaceum may be stored at a temperature in a range of approximately −20° C. to approximately 10° C. and at a relative humidity in a range of approximately 90% to approximately 95%.

A second embodiment of the present invention describes a method for obtaining a peel extract of Nephelium lappaceum. The method comprises grinding a fresh shell of the Nephelium lappaceum to create a material, where a particle size of the material is in a range of approximately 100 microns to approximately 1000 microns.

Next, the method includes cold pressing the material. The cold pressing of the material may occur at a pressure in a range of approximately 1000 psi to approximately 3000 psi until all water in the shell is extracted. Subsequent to the cold pressing of the material, the method may include removing the material from the cold press, adding approximately 95% of a solvent to the material, and allowing the material to rest for a time period in a range of approximately 5 minutes to approximately 10 minutes. This process may be repeated approximately 3 times for maximum extraction yield. In examples, the solvent may be ethanol, ethyl acetate, acetone, 1-butanol, methanol, 1-propanol, and/or water, among others not explicitly listed herein.

Next, the method may include collecting an aqueous extract from the material and stabilizing the aqueous extract with a first acid until a first predetermined pH is reached. The first acid may include citric acid, malic acid, or another food grade organic acid. Then, the method includes stabilizing the material with a second acid until a second predetermined pH is reached. The second acid may include citric acid, phosphoric acid, malic acid, acetic acid, or lactic acid. Each of the first predetermined pH and the second predetermined pH is a pH of 4.6 or below, but not less than a pH of 2.5. In examples, the second determined pH is greater than the first determined pH to form a coacervation during encapsulation. In other examples, the second determined pH is a pH of 4.6. The method may then include concentrating the aqueous extract and obtaining a powder extract or a liquid extract of the shell of the Nephelium lappaceum via a means.

It should be appreciated that the means to obtain the powder extract of the shell of the Nephelium lappaceum may include freeze drying, spray drying, or spray freeze drying and the means to obtain the liquid extract of the shell of the Nephelium lappaceum may include high temperature short time (HTST) pasteurization, ultra-high temperature (UHT) processing, or aseptic processing. The shell or peel extract of the Nephelium lappaceum may be utilized in a food composition, a beverage composition, and/or a supplement composition.

A third embodiment of the present invention describes a formulation for delivering one or more health benefits to a human post-consumption. Each of the one or more health benefits to the human include support or regulation of blood sugar, blood pressure, body weight, performance/recovery from sports, immune health, gut health, neuroprotectice effect, anti-aging, pain management, and/or joint health. The formulation includes a shell extract of Nephelium lappaceum in a form of a powder or a liquid and at least one material (e.g., encapsulating material) selected from the group consisting of: a polysaccharide, a protein, a lipid, and a food grade compound. In some examples, the ratio of the shell extract of Nephelium lappaceum to the at least one material is in a range between 1:1 to 1:50. In other examples, the ratio of the shell extract of the Nephelium lappaceum to the at least one material is 50:1. In further examples, the ratio of the shell extract of the Nephelium lappaceum to the at least one material is 10:1.

In some examples, the formulation is added to a carrier prior to consumption by the human. In other examples, the formulation is encapsulated prior to consumption by the human. A method to prepare the encapsulated formulation may include: dissolving or suspending the at least one material in water at a temperature in a range between approximately 25° C. to approximately 100° C., stirring the at least one material to keep the at least one material in a solution or suspended for a time period in a range between approximately 15 minutes to approximately 90 minutes, and passing the formulation through a device to form a micro-nano encapsulation of the formulation.

In response to a determination that the micro-nano encapsulation of the formulation is required in a powder form, the method may include subjecting the micro-nano encapsulation of the formulation to a means, such as freeze drying, spray drying, vacuum drying, solar drying, or spray freeze drying. In response to a determination that the micro-nano encapsulation of the formulation is required in a liquid form, the method may include subjecting the micro-nano encapsulation of the formulation to a means, such as an ultrasonic means or a microfluidizer means, and further the HTST pasteurization or the UHT processing. The micro-nano encapsulation of the formulation may be stored in a container.

In general, the present invention succeeds in conferring the following benefits and objectives.

It is an object of the present invention to provide formulations for delivering the one or more health benefits to the human post-consumption.

It is an object of the present invention to provide formulations for delivering the one or more health benefits to the human post-consumption that comprises the shell extract of the Nephelium lappaceum (or rambutan) in the form of the powder or the liquid and the at least one material (e.g., encapsulating material).

It is an object of the present invention to provide the formulations for delivering the one or more health benefits to the human post-consumption that comprises the shell extract of Nephelium lappaceum (or rambutan) in the form of the powder or the liquid and the at least one material (e.g., encapsulating material), where the at least one material includes a polysaccharide, a protein, a lipid, and/or a food grade compound.

It is an object of the present invention to provide the formulations for delivering the one or more health benefits to the human post-consumption, where the one or more health benefits include support or regulation of blood sugar, blood pressure, body weight, performance/recovery from sports, immune health, gut health, neuroprotectice effect, anti-aging, pain management, and/or joint health.

### BRIEF DESCRIPTION OF THE DRAWINGS

FIG. 1 depicts a block diagram of a method to maintain or preserve a freshness of a shell of Nephelium lappaceum during a non-harvest season, according to at least some embodiments described herein.

FIG. 2 depicts a block diagram of a method for obtaining a peel extract of Nephelium lappaceum, according to at least some embodiments described herein.

FIG. 3 depicts a block diagram of a method for preparing a formulation that is encapsulated and delivers one or more health benefits to a human post-consumption, according to at least some embodiments described herein.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, “a formulation” may be used interchangeably with “a composition” and/or “a supplement,” throughout the specification and refer to the composition as described herein for delivering the one or more health benefits to the human post-consumption. The supplement may be in any form, including a solid form (e.g. a powder), a semi-solid form (e.g. a food-like consistency/gel), or a liquid form. Further, the supplement may be in the form of a tablet or a capsule. The liquid can conveniently be mixed in with food or ingested directly (e.g., via a spoon or via a pipette-like device). The supplement may be high in one or more components of the invention or may be in the form of a combined pack of at least two parts, each part containing the required level of one or more component.

In certain embodiments, the disclosed formulation can be in the form of a concentrate that is diluted prior to use. In certain embodiments, the formulation can be supplemented with a pharmaceutical composition.

Under certain conditions, oxygen can have serious deleterious effects on the body. For example, oxygen can produce highly reactive compounds called reactive oxygen species (ROS). ROS are a type of unstable molecule that contain oxygen and that easily react with other molecules in a cell. Examples of ROS include peroxides, superoxide, hydroxyl radical, singlet oxygen, and alpha-oxygen.

Hydroxyl radical (OH), the superoxide radical (O2), the nitric oxide radical (NO) and the lipid peroxyl radical (LOO) are amongst the most important oxygen-containing radicals formed in the body. Free radicals can attack a number of macro-molecules including lipids, proteins, and DNA, resulting in the cellular damage. See, V. Hajhashemi, et al., “Are antioxidants helpful for disease prevention?,” Res. Pharm. Sci, 2010, 5(1), Pages 1-8; and Marian Valko, et al., “Free Radicals and Antioxidants in Normal Physiological Functions and Human Disease,” Int. J. Biochem. Cell Biol., 2007, 39(1), Pages 44-84.

Free radicals and other ROS are derived either from the enogenous metabolic processes in the human body (such as mitochondrial respiration, peroxisomal metabolism, phagocyte activity, arachidonate pathways, inflammation, ischaemia, exercise, and reactions involving iron and other transition metals) or from external sources (such as exposure to radiation, ozone, cigarette smoke, air pollutant, and industrial chemicals). See, L. Langseth, “Oxidants, antioxidants and disease prevention,” 1996, 16, Pages 840-841.

Antioxidants are molecules that can safely react with these free radicals to neutralize or terminate the chain reaction before vital molecules are damaged. As such, antioxidants are important in numerous fields and represent a line of defense against major health conditions, including heart disease, cancer, and muscular degeneration. See, Silvia Vertuani, et al., “The Antioxidants and Pro-Antioxidants Network: An Overview,” Curr. Pharm. Des., 2004, 10(14), Pages 1677-16694.

Moreover, the human body may gain antioxidants from superoxide dismutase, catalase, and glutathione or from ingesting dietary antioxidants, from sources such as fruit, vegetables, nuts, and grains. As one ages, ones body's defense system loses its effectiveness and therefore holds fewer antioxidants. To reduce the damage to ones bodies caused by free radicals, one needs to add antioxidants to his/her diet.

Nephelium lappaceum, or rambutan, is an edible fruit produced by a tropical tree in the family Sapindaceae. The rambutan is native to Southeast Asia and is closely related to several other edible tropical fruits including the lychee, longan, pulasan, and mamoncillo. Rambutan is rich in many vitamins and minerals. More specifically, the rambutan peel and its seed are thought to be rich sources of nutrients, antioxidants and other beneficial compounds. See, Cristian Hernández, et al., “Polyphenolic Content, in Vitro Antioxidant Activity and Chemical Composition of Extract From Nephelium lappaceum L. (Mexican Rambutan) Husk,” Asian Pac. J. Trop. Med., 2017, 10(12), Pages 1201-1205; and Yongliang Zhuang, et al., “Protective Effects of Rambutan (Nephelium lappaceum) Peel Phenolics on H 2 O 2-induced Oxidative Damages in HepG2 Cells and D-Galactose-Induced Aging Mice,” Food Chem. Toxicol., 2017, 108 (Pt B), Pages 554-562. Additionally, the flesh of rambutan is proven to comprise a range of approximately 1.3 grams to approximately 2 grams of total fiber per 3.5 ounces (100 grams). See, S. Gorinstein, et al., “Comparative Content of Total Polyphenols and Dietary Fiber in Tropical Fruits and Persimmon,” J. Nutr. Biochem., 1999, 10(6), Pages 367-371.

Further, the peel of rambutan has been found to be useful as a source of natural antioxidants for food or drug product because of its high antioxidant activity and non-toxic property to normal cells. See, Siriporn Okonogi, et al., “Comparison of antioxidant capacities and cytotoxicities of certain fruit peels,” Food Chemistry, 2007, 103(3), Pages 839-846; Suganya Tachakittirungrod, et al., “Study on antioxidant activity of certain plants in Thailand: Mechanism of antioxidant action of guava leaf extract,” Food Chemistry, 2007, 103(2), Pages 381-388; and L. P. Leong, et al., “An investigation of antioxidant capacity of fruits in Singapore markets,” Food Chemistry, 2002, 76(1), Pages 69-75. Some of these studies have found that rambutan peel phenolic (RPP) extracts can be used as a natural antioxidant and anti-aging agent in the pharmaceutical and food industries. See, Yongliang Zhuang, et al., “Protective Effects of Rambutan (Nephelium lappaceum) Peel Phenolics on H 2 O 2-induced Oxidative Damages in HepG2 Cells and D-Galactose-Induced Aging Mice,” Food Chem. Toxicol., 2017, 108 (Pt B), Pages 554-562.

In view of these findings, the instant invention describes a formulation that may deliver one or more health benefits to the human post-consumption. The one or more health benefits to the human may include support or regulation of blood sugar, blood pressure, body weight, performance/recovery from sports, immune health, anti-aging, pain management, and/or joint health.

According to an example, the formulation includes a shell extract of Nephelium lappaceum in a form of a powder or a liquid and at least one material (e.g., encapsulating material). The at least one material includes a polysaccharide, a protein, a lipid, and/or a food grade compound.

In examples, the polysaccharide may be guar gum, cyclodextrin, maltodextrin, xanthan gum, gum arabic, gellan gum, pectin, chitosan, modified starches, soy gum, cellulose phthalate acetate, cellulose acetate butyrate, ethyl cellulose, and/or acetyl cellulose, among others not explicitly listed herein. The protein may include gelatin, casein, whey protein, and/or protein isolated from peas, zein, collagen, fungus, mycoproteins, almond, chickpeas, lentils, rice, beans, kale, quinoa, and/or soy, among others not explicitly listed herein. The lipid may include lecithin, glycerides, polyglycerides, and/or liposomes, among others not explicitly listed herein. In some examples, other carriers may be propilenglicol, polipropilenglicol, magnesium stearate, and/or silicon dioxide, among others not explicitly listed herein.

In an example, an aliquot of the obtained shell extract of Nephelium lappaceum is taken (aqueous fraction and the ethanol fraction) to determine an amount of dissolved solids in each one. The aliquot is brought to dryness using the rotavapor and subsequently dried. Once dry, the mass is determined. The mass value obtained is used to determine the amount of the at least one material (e.g., encapsulating material).

In some examples, the ratio of the shell extract of Nephelium lappaceum to the at least one material (e.g., the encapsulating material) is in a range between 1:1 to 1:50. In other examples, the ratio of the shell extract of Nephelium lappaceum to the at least one material (e.g., the encapsulating material) is 50:1. In another example, the ratio of the shell extract of Nephelium lappaceum to the at least one material (e.g., the encapsulating material) is 10:1. In additional examples, the shell extract of Nephelium lappaceum comprises 40% -90% of polyphenols in a way that the encapsulating micro-nano material is in a 1:1 ratio with the polyphenols.

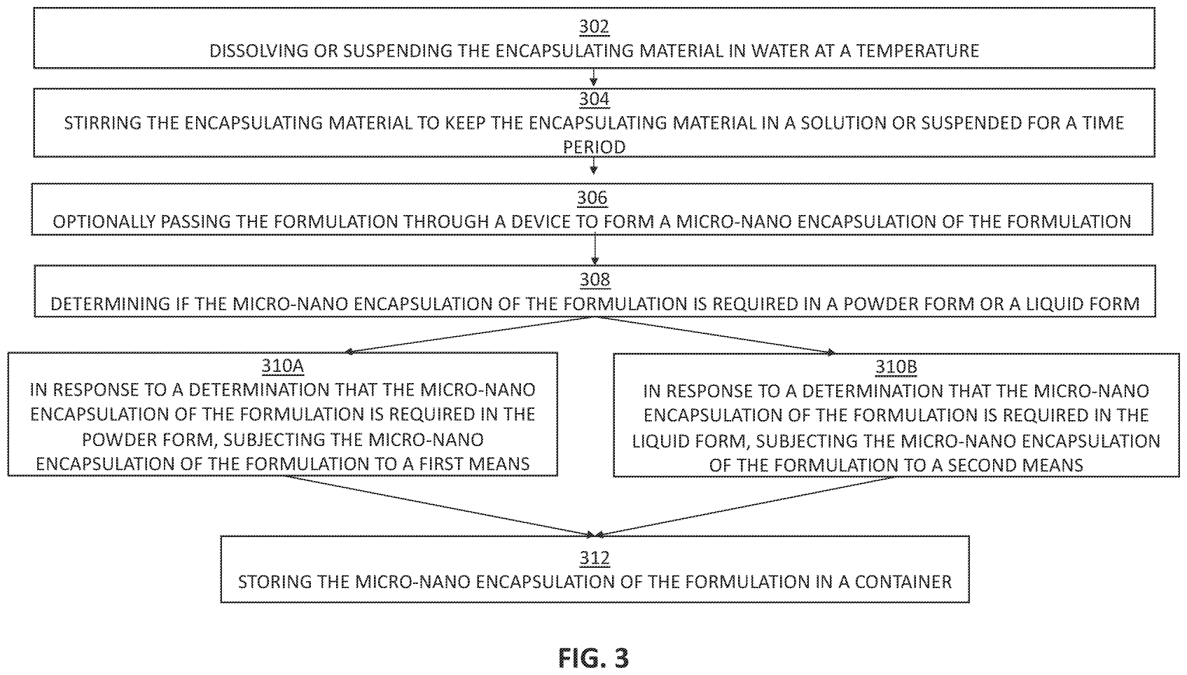

In some examples, the formulation is added to a carrier prior to consumption by the human. In other examples, the formulation is encapsulated prior to consumption by the human. The method of encapsulation is described in FIG. 3.

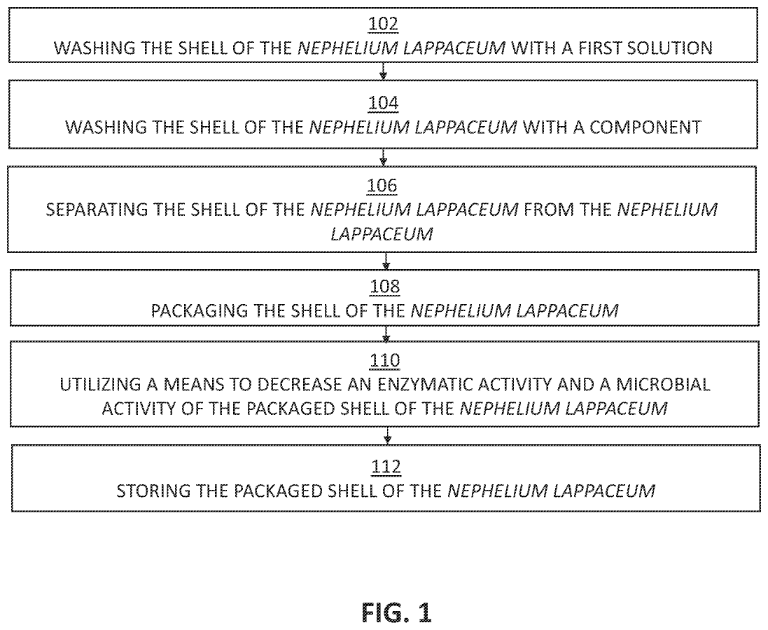

FIG. 1 depicts a block diagram of a method to maintain or preserve a freshness of a shell or a peel of Nephelium lappaceum during a non-harvest season, according to at least some embodiments described herein. As depicted in FIG. 1, the method to maintain or preserve the freshness of the shell or the peel of Nephelium lappaceum during the non-harvest season comprises numerous process steps. The method of FIG. 1 may begin at a process step 102 that includes washing the shell of the Nephelium lappaceum with a first solution (e.g., a neutral soapy solution). A process step 104 may occur subsequent the process step 102 and may include washing the shell of the Nephelium lappaceum with a component (e.g., a 100 parts per million chlorine solution, quaternary ammonium compounds, acetic acid, peracetic acid, or other non-toxic disinfectant). A process step 106 may occur subsequent the process step 104 and may include separating the shell of the Nephelium lappaceum from the Nephelium lappaceum.

A process step 108 may occur subsequent the process step 106 and may include packaging the shell of the Nephelium lappaceum via vacuum packaging or another similar method. In some examples, the shell of the Nephelium lappaceum may be vacuum packaged with a coating. In examples, the shell of the Nephelium lappaceum may be vacuum packaged with polyamide or polyethylene in different and opaque coatings that avoids contact with light.

A process step 110 may occur subsequent the process step 108 and may include utilizing a means to decrease an enzymatic activity and a microbial activity of the packaged shell of the Nephelium lappaceum. The means to decrease the enzymatic activity and the microbial activity of the packaged shell of the Nephelium lappaceum may include freeze shocking with air, pressurized carbon dioxide, or liquid nitrogen. It should be appreciated that the process step 108 and the process step 110 may occur in a reverse order (e.g., the process step 110 occurs before the process step 108). In another example, one of the process step 108 and the process step 110 may not be used.

A process step 112 may occur subsequent the process step 110 and may include storing the packaged shell of the Nephelium lappaceum at a temperature in a range of approximately −20° C. to approximately 10° C. and at a relative humidity in a range of approximately 90% to approximately 95%. The process step 112 may conclude the method to maintain the freshness of the shell of Nephelium lappaceum of FIG. 1.

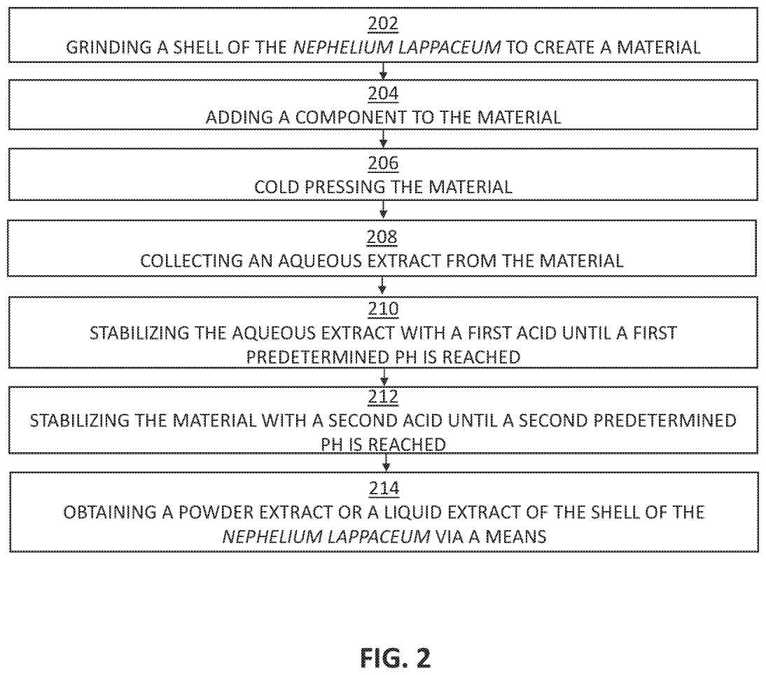

FIG. 2 depicts a block diagram of a method for obtaining a peel extract of Nephelium lappaceum, according to at least some embodiments described herein. The method for obtaining the peel extract of the Nephelium lappaceum of FIG. 2 includes numerous process steps. The method for obtaining the peel extract of the Nephelium lappaceum of FIG. 2 may begin at a process step 202 that includes grinding a shell of the Nephelium lappaceum to create a material, where a particle size of the material is in a range of approximately 100 microns to approximately 1000 microns. The process step 202 may occur via a device, such as a rotary mill, a cutter, a bowl chopper, and/or a blender, among other similar devices. A process step 204 follows the process step 202 and includes adding a component to the material, such as a nitrogen gas, an ascorbic acid, or a citric acid to prevent oxidation of active components or enzymes.

A process step 206 follows the process step 204 and includes cold pressing the material. The cold pressing of the material may occur at a pressure in a range of approximately 1000 psi to approximately 3000 psi until all water in the shell is extracted. The process step 206 may also be assisted through use of supercritical fluid, ultrasound, microwave, and/or pressurized liquid including several, combinations, or just one of the methods or none, previously washed with a food grade disinfectant. It should be appreciated that “supercritical fluid” is any substance at a temperature and pressure above its critical point, where distinct liquid and gas phases do not exist. It can effuse through solids like a gas, and dissolve materials like a liquid.

Subsequent to the cold pressing of the material of the process step 206, the method may include removing the material from the cold press, adding approximately 95% of a solvent to the material, and allowing the material to rest for a time period in a range of approximately 5 minutes to approximately 10 minutes. In examples, the solvent may be ethanol, ethyl acetate, acetone, 1-butanol, methanol, 1-propanol, and/or water, among others not explicitly listed herein. In some examples, subsequent the process step 206 and prior to the process step 208, the material may be cold pressed again. It should be appreciated that in other examples, the process step 206 may occur at least two times, three times, four times, or five times.

A process step 208 follows the process step 206 and includes collecting an aqueous extract from the material. In some examples, the aqueous extract of FIG. 2 is an ethanolic extract. In other examples, the aqueous extract of FIG. 2 is another extract. A process step 210 follows the process step 208 and includes stabilizing the aqueous extract with a first acid until a first predetermined pH is reached. The first acid may include citric acid, malic acid, or another food grade organic acid. The first predetermined pH is a pH of 4.6 or below, but not less than a pH of 2.5. The process step 210 may also include concentrating the aqueous extract and bringing the aqueous extract to a minimum possible volume.

A process step 212 follows the process step 210 and includes stabilizing the material with a second acid until a second predetermined pH is reached. The second acid may include citric acid, phosphoric acid, malic acid, acetic acid, or lactic acid. The second predetermined pH is a pH over 4.6. The first determined pH is modified to the second determined pH to form a coacervation that leads to an encapsulation.

A process step 214 follows the process step 212 and includes obtaining a powder extract or a liquid extract of the shell of the Nephelium lappaceum via a means. It should be appreciated that the means to obtain the powder extract of the shell of the Nephelium lappaceum may include freeze drying, spray drying, or spray freeze drying. The means to obtain the liquid extract of the shell of the Nephelium lappaceum may include ultrasonic methods/means, microfluidizer methods/means, high temperature short time (HTST) pasteurization methods/means, ultra-high temperature (UHT) processing, or aseptic processing in a time frame between approximately 2 seconds to approximately 60 seconds and at a temperature in a range between approximately 70° C. and approximately 170° C. In some examples, the liquid extract of the shell of the Nephelium lappaceum may be obtained in a range between 14% to 22% of total solids. The shell extract of the Nephelium lappaceum of FIG. 2 may be utilized in a food composition, a beverage composition, and/or a supplement composition.

FIG. 3 depicts a block diagram of a method for preparing a formulation that is encapsulated and delivers one or more health benefits to a human post-consumption, according to at least some embodiments described herein.

The method for preparing the formulation that is encapsulated and delivers the one or more health benefits to the human post-consumption is depicted in FIG. 3 and includes numerous process steps. The method of FIG. 3 may begin at a process step 302 that includes dissolving or suspending the at least one material in water at a temperature in a range between approximately 25° C. to approximately 70° C. In some examples, the temperature of the process step 302 is in the range between approximately 25° C. to approximately 45° C. In other examples, the process step 302 is assisted by emulsifiers, surfactants, thickeners, and/or other components.

The process step 302 may be followed by a process step 304 that includes stirring the at least one material between 100 rpm and 1500 rpm to keep the at least one material in a solution or suspended for a time period in a range between approximately 15 minutes to approximately 90 minutes. In some examples, the time period of the process step 304 is in the range between approximately 25 minutes to approximately 60 minutes. In examples, the process step 302 may be assisted by sonication, microwaving, microfluidic means/methods, and/or supercritical fluid, among other methods or means. In some examples, the process step 302 and the process step 304 may be repeated a quantity of times depending on a quantity of layers.

An optional process step 306 follows the process step 304 and includes passing the formulation through a device to form a micro-nano encapsulation of the formulation. In some examples, the device is a cavitator or microfluidizer and the formulation is passed through the cavitator at a power in a range between approximately 20 Watts and approximately 16000 Watts, at a frequency in a range between approximately 20 KHz and approximately 60 KHz, for a time period in a range between approximately 0.05 minutes and approximately 60 minutes, and at an amplitude in a range between approximately 40 microns and approximately 120 microns. It should be appreciated that some layers of the micro-nano encapsulation of the formulation may be assisted by spray coating, fluid bed, film coating, and/or spheronification, among other means or methods. The optional process step 306 may not be required when the mixed material enhances the micro or nano formulations. The optional process step 306 may be followed by an optional process step including identifying if the micro-nano encapsulation of the formulation has more than one layer, and if so, centrifuging the micro-nano encapsulation of the formulation between approximately 10,000 rpm and approximately 15,000 rpm, for the time period between approximately 10 minutes and approximately 30 minutes, and between the temperature of approximately 4° C. and approximately 25° C.

A process step 308 follows the process step 306 and includes determining if the micro-nano encapsulation of the formulation is required in a powder form or a liquid form. In response to a determination that the micro-nano encapsulation of the formulation is required in a powder form, the process step 308 is followed by a process step 310A that includes subjecting the micro-nano encapsulation of the formulation to a means, such as freeze drying, spray drying, vacuum drying, solar drying, or spray freeze drying. In response to a determination that the micro-nano encapsulation of the formulation is required in a liquid form, the process step 308 is followed by a process step 310B that includes subjecting the micro-nano encapsulation of the formulation to a means, such as sonicator or microfluidizer and the HTST pasteurization or the UHT processing, as described in FIG. 2.

The process step 310A or the process step 310B may be followed by a process step 312 that includes storing the micro-nano encapsulation of the formulation in a container. The process step 312 may include storing the container in an inert atmosphere or in a vacuum that protects it from light. The process step 312 concludes the method for preparing the formulation that is encapsulated and delivers the one or more health benefits to the human post-consumption is depicted in FIG. 3.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for obtaining a peel extract of Nephelium lappaceum, the method comprising:
grinding a shell of the Nephelium lappaceum to create a material;
adding a component to the material;
cold pressing the material;
collecting an aqueous extract from the material;
stabilizing the aqueous extract with a first acid until a first predetermined pH is reached;
stabilizing the material with a second acid until a second predetermined pH is reached;
concentrating the aqueous extract; and
obtaining a powder extract or a liquid extract of the shell of the Nephelium lappaceum via a means.

2. The method of claim 1, wherein the shell extract of the Nephelium lappaceum is utilized in a food composition, a beverage composition, or a supplement composition.

3. The method of claim 1, wherein a particle size of the material is in a range of approximately 100 microns to approximately 1000 microns.

4. The method of claim 1, wherein the component is selected from the group consisting of: a nitrogen gas, an ascorbic acid, and a citric acid.

5. The method of claim 1, wherein the cold pressing of the material occurs at a pressure in a range of approximately 1000 psi to approximately 3000 psi until all water in the shell is extracted.

6. The method of claim 1, further comprising:
subsequent to the cold pressing of the material,
removing the material from the cold press;
adding approximately 95% of a solvent to the material; and
allowing the material to rest for a time period in a range of approximately 5 minutes to approximately 10 minutes.

7. The method of claim 1, wherein the solvent is selected from the group consisting of: ethanol, ethyl acetate, acetone, 1-butanol, methanol, 1-propanol, and water.

8. The method of claim 1,
wherein the first acid is selected from the group consisting of: citric acid, malic acid, and another food grade organic acid,
wherein the second acid is selected from the group consisting of: citric acid, phosphoric acid, malic acid, acetic acid, and lactic acid,
wherein each of the first predetermined pH and the second predetermined pH is in a range of a pH of 2.5 to a pH of 4.6, and
wherein the second determined pH is greater than the first determined pH to form a coacervation during encapsulation.

9. The method of claim 1,
wherein the means to obtain the powder extract of the shell of the Nephelium lappaceum is selected from the group consisting of: freeze drying, spray drying, and spray freeze drying, and
wherein the means to obtain the liquid extract of the shell of the Nephelium lappaceum is selected from the group consisting of: high temperature short time (HTST) pasteurization, ultra-high temperature (UHT) processing, and aseptic processing.
